(19) United States
(12) Patent Application Publication
(10) Pub. No.: US 2011/0295337 A1
(43) Pub. Date: Dec. 1, 2011

(54) Systems and Methods For Regulating Metabolic Hormone Producing Tissue

(76) Inventors: Thomas E. Albrecht, Cincinnati, OH (US); Jeffrey L. Aldridge, Lebanon, OH (US); Edward G. Chekan, Cincinnati, OH (US); Sean P. Conlon, Loveland, OH (US); Michael S. Cropper, Edgewood, KY (US); Daniel F. Dlugos, JR., Middletown, OH (US); Jason L. Harris, Mason, OH (US); Christopher J. Hess, Cincinnati, OH (US); Kevin L. Houser, Springsboro, OH (US); John V. Hunt, Cincinnati, OH (US); Gary L. Long, Cincinnati, OH (US); Prasanna Malaviya, Mason, OH (US); Amy L. Marcotte, Mason, OH (US); Rudolph H. Nobis, Mason, OH (US); Mark S. Ortiz, Milford, OH (US); Mark D. Overmyer, Cincinnati, OH (US); Alessandro Pastorelli, Roma (IT); David N. Plescia, Cincinnati, OH (US); Galen C. Robertson, Durham, NC (US); Randy J. Seeley, Cincinnati, OH (US); Frederick E. Shelton, IV, Hillsboro, OH (US); Michael J. Stokes, Cincinnati, OH (US); Foster B. Stulen, Mason, OH (US); Richard W. Timm, Cincinnati, OH (US); James W. Voegele, Cincinnati, OH (US); William B. Weisenburgh, II, Maineville, OH (US); James A. Woodard, JR., Mason, OH (US); David C. Yates, West Chester, OH (US); Andrew M. Zwolinski, Hamburg (DE)

(21) Appl. No.: 13/105,262

(22) Filed: May 11, 2011

Related U.S. Application Data
(60) Provisional application No. 61/348,273, filed on May 26, 2010.

(51) Int. Cl. A61N 1/36

(52) U.S. Cl. 607/40

(57) ABSTRACT

A method for regulating hormone production comprises placing at least one electrode in a gastrointestinal tract of a patient and recording an electrical signal during a preselected event produced by the gastrointestinal tract. The method further involves the steps of storing the electrical signal, and playing back the electrical signal by activating the electrode during the absence of the preselected event.

### PRIORITY

This application claims priority to U.S. Provisional Patent Application Ser. No. 61/348,273, entitled “Systems and Methods for Regulating Metabolic Hormone Producing Tissue,” filed May 26, 2010, the disclosure of which is incorporated by reference herein.

### FIELD OF THE INVENTION

This invention relates to systems, devices, and methods for the control of obesity. In particular, this invention relates to regulation of ghrelin and other hunger controlling hormones in the gastrointestinal tract through the application of electric potential.

### BACKGROUND

Ghrelin is a hormone produced by the endocrine or oxyntic in the stomach and is one of the key mediators within the circulatory response for the regulation of body weight. Therefore, there is a need for methods and devices to control levels of hormones (e.g. ghrelin) released into the blood stream and in turn control appetite. In addition to ghrelin, the endogenously produced control substance can be selected from one or more of the group consisting of gastrin, somatostatin, secretin, cholecystokinin (CCK), a CCK analog, a CCK receptor agonist, incretins, GLP-1, GIP, DDP-4, ghrelin, ghrelin antagonist, leptin, neuropeptide Y, peptide YY (PYY), a PYY analog, GLP-1, a GLP-1 analog, oxyntomodulin, cortisol, deoxycorticosterone, flurohydrocortisone, beclomethasone, betamethasone, cortisone, dexamethasone, fluocinolone, fluocinonide, fluocortolone, fluorometholone, fluprednisolone, flurandrenolide, halcinonide, hydrocortisone, medrysone, methylprednisolone, paramethasone, prednisolone, prednisone, triamcinolone, danazole, fluoxymesterone, mesterolone, dihydrotestosterone methyltestosterone, testosterone, dehydroepiandrosetone, dehydroepiandrostendione, calusterone, nandrolone, dromostanolone, oxandrolone, ethylestrenol, oxymetholone, methandriol, stanozolol methandrostenolone, testolactone, cyproterone acetate, diethylstilbestrol, estradiol, estriol, ethinylestradiol, mestranol, quinestrol chlorotrianisene, clomiphene, ethamoxytriphetol, nafoxidine, tamoxifen, allylestrenol, desogestrel, dimethisterone, dydrogesterone, and combinations thereof.

### SUMMARY OF THE INVENTION

The present invention provides systems and methods for controlling the production and release of ghrelin by the application of electric potential.

In a first series of embodiments, ghrelin and other hunger hormones are regulated by electrical pulses applied to control secretion levels of the hormone through the action of reversible electroporation. Periodic DC pulses are applied to tissue to control the secretion of the hormone through control of plasma membrane potential (PMP), either though depolarizing or hyperpolarizing of the cell. In one embodiment, one or more pulses are applied using electrodes.

In a second series of embodiments, sensing and control systems are presented to activate pulses according to natural digestive cycles. Methods for regulating pulse application time and power consumption are disclosed including the use of biofeedback mechanisms.

In a third series of embodiments, methods and devices are described to provide power for pulsing to control hormone (e.g. ghrelin) secretion levels.

And in a fourth series of embodiments, methods and systems for preventing the dislodging of control and power systems are described with respect to the aforementioned embodiment sets. Mounting and fixture devices are described as examples.

### DESCRIPTION OF THE FIGURES

The invention will be more fully understood from the following detailed description taken in conjunction with the accompanying drawings in which:

FIG. 1 is a schematic diagram of the system of the present disclosure.

FIG. 2A is a top view of a stomach with a first electrode system.

FIG. 2B is a top view of a stomach with a second electrode system.

FIG. 2C is a top view of a stomach with a third electrode system.

FIG. 3A is a top view of a patient esophagus fitted with a valve and sensor device.

FIG. 3B is a top view of a patient esophagus fitted with a valve and sensor device.

FIG. 4 is a top view of a patient fitted with a device for metabolic hormone feedback.

FIG. 5A is a top view of a stomach fitted with a first example of a stomach stretch sensor.

FIG. 5B is a top view of stomach fitted with a second example of a stomach stretch sensor.

FIG. 6 is graph relating pH within the stomach to the time of day.

FIG. 7 is a top view of a stomach including a sensor.

FIG. 8 is a schematic diagram of a gastric sensor in a stomach with an external receiving unit.

FIG. 9 is an illustration of a method of assembly of a flexible biosensor.

FIG. 10 is a side view of an implanted submucosal sensor.

FIG. 11 is a schematic view of an external power magnet.

FIG. 12 is a top view of a patient having an implanted subcutaneous photovoltaic cell.

FIG. 13A is a top view of a stomach having a gastric coil.

FIG. 13B is a perspective view of energy generating petals in a first example.

FIG. 13C is a perspective view of energy generating petals in a second example.

FIG. 13D is a perspective view of energy generating petals in a third example.

FIG. 13E is a perspective view of energy generating petals in a fourth example.

FIG. 14 is a side view of power generating mass.

FIG. 15 is a perspective view of an implanted peltier device.

FIG. 16 is a perspective view of a coil having a sliding magnet.

FIG. 17 is an expanded view of an implanted gastric coil.

FIG. 18A is a perspective view of a first embodiment of a belly ball.

FIG. 18B is a perspective view of a second embodiment of a belly ball.

FIG. 19 is a side view of folded Hoberman sphere.

FIG. 20 is a perspective view of a spherical flex-ribbed structure.

FIG. 21 is a perspective view of an oblong flex-ribbed structure.

FIG. 22A is a perspective view of an example of a stomach wall anchor system.

FIG. 22B is a perspective view of an example of a stomach wall anchor system.

FIG. 22C is a perspective view of an example of a stomach wall anchor system.

FIG. 22D is a perspective view of an example of a stomach wall anchor system.

FIG. 22E is a perspective view of an example of a stomach wall anchor system.

FIG. 22F is a perspective view of an example of a stomach wall anchor system.

### DETAILED DESCRIPTION

The present invention discloses systems, methods, and devices for controlling the production and release of ghrelin and/or other hunger controlling hormones by the application of electric potential to induce reversible electroporation. Exemplary embodiments herein are described to provide an overall understanding of the principles, structure, function, manufacture, and uses of the systems, methods, and devices as disclosed. Many specific examples of these embodiments are illustrated in the accompanying Figures (Figs.). Those skilled in the art will understand that the systems, devices, and methods described herein and illustrated in the accompanying Figures are non-limiting exemplary embodiments and that the scope of the present invention is defined solely by the claims. The features illustrated or described with respect to one exemplary embodiment may be combined with features of other embodiments described herein. Such modifications and variations are intended to be within the scope of the present invention as described. Subject matter disclosed herein is related to subject matter disclosed in PCT Application WO 2008/028108 A2 and also subject matter in U.S. patent application Ser. No. 12/261,079 assigned to Ethicon Endo-Surgery, Inc., the complete disclosures of which are incorporated herein by reference.

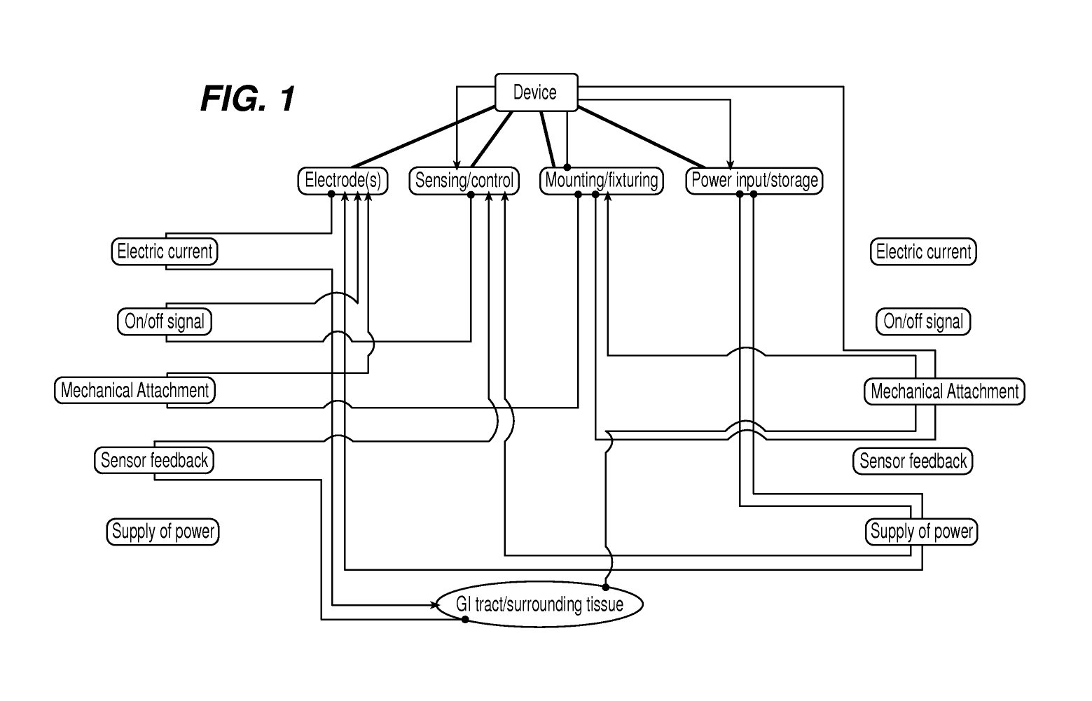

In a first series of embodiments, ghrelin and/or other hunger controlling hormones are regulated by electrical pulses applied to control secretion levels of the hormone. Periodic DC pulses are applied to tissue to control the secretion of the hormone through control of the plasma membrane potential (PMP) of the cell either though depolarizing or hyperpolarizing of the cell. By modifying the PMP, forced reversible poration of targeted cells will be induced in order to facilitate the crossing of metabolic controlling substances over the cell membrane. In one embodiment, the intensity of the electric field would be set to forcibly and reversibly create pores in cells matched in size to the molecule of selected metabolic controlling hormones. The system would be calibrated to administer sufficient electric potential to the target cells to ensure that pores are induced in the cells that allow the passage of a desired hunger controlling hormone molecule. Multiple sites could administer multiple magnitudes of electric potential to allow the release of multiple sizes of hunger controlling hormone molecules. In another embodiment, one or more pulses of electric DC are applied using electrodes. In another embodiment, the applied electric potential is limited to the range of 0.0 to 1.0 volts DC to reduce the likelyhood of irreversible electroporation of the target cells. In a second series of embodiments of the second sub-system, sensing and control systems are presented to activate pulses according to natural digestive cycles. Methods for regulating pulse application time and power consumption are also disclosed including the use of biofeedback mechanisms. In a third series of embodiments of the third sub-system, methods and devices are described to provide power for pulsing in the control of secretion levels of ghrelin. And in a fourth series of embodiments for the fourth sub-system, methods and systems for preventing dislodging of the control and power systems are described with respect to the aforementioned embodiment sets. Mounting and fixture devices are described as examples. Turning now to FIG. 1, a system for controlling the production and release of ghrelin and/or other hunger hormones by the application of electric potential is shown comprising four sub-systems. The first sub-system involves electrical stimulation using an electrode-based system wherein electrical pulses are applied to control secretion levels of ghrelin and/or other hunger hormones. The second sub-system includes systems and methods for sensing and control wherein the system may activate pulses according to natural digestive cycles of a patient. The third sub-system is presented to provide power for the pulse system. The fourth sub-system includes methods and systems for mounting and fixing the various components presented herein and to prevent dislodging of the electrodes, and the control and power systems. The electrode pulse system of the present invention offers several benefits. The systems and methods described herein offer a reversible and patient customizable means to cause extended satiety. Further, the technology of the present invention may be implemented through minimally-invasive endoscopic or laparoscopic means.

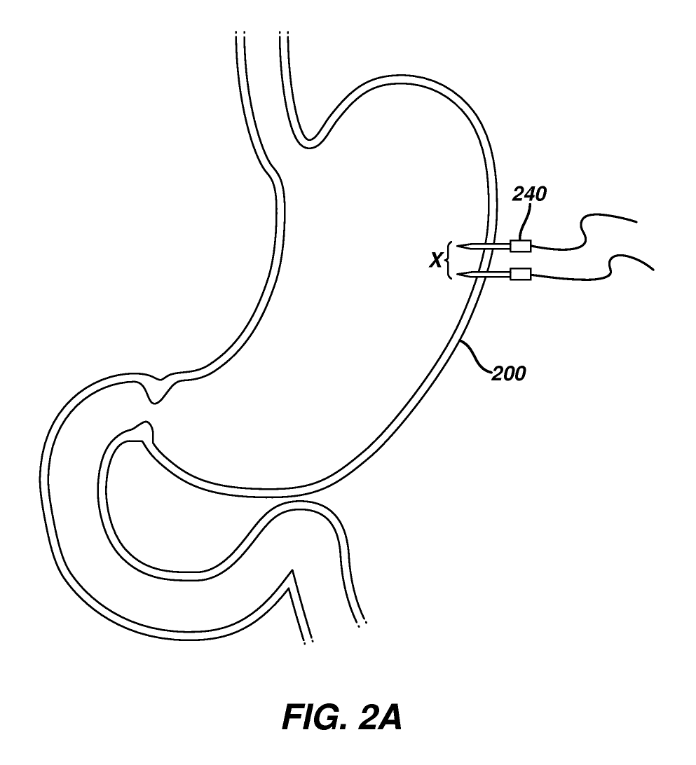

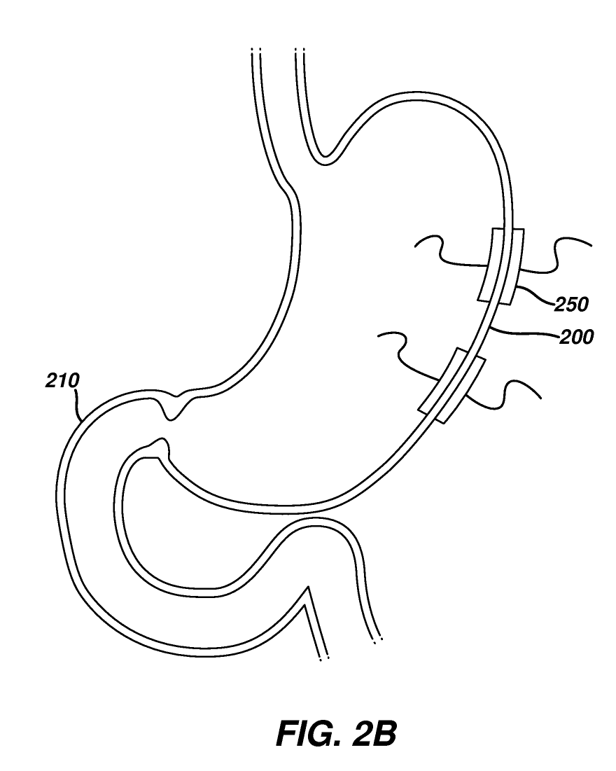

As seen in FIG. 2A, an embodiment of the first sub-system is shown wherein a two-electrode spike 240 is imbedded in an area of tissue of a stomach 200 that is desirable to control. Depth of penetration of the two-electrode spike 240 may include one or multiple layers of the stomach or intestinal tissue. These may be held in place by suture, stapling or any other suitable fastening means. A staple or clip may be used as an electrode. Multiple electrode surfaces on each staple may be used as electrode contact surfaces. The two-electrode spike 240 is used to emit DC pulses to the tissue to control secretion of ghrelin and/or other hunger hormones. Secretion is controlled by regulation of the plasma membrane potential (PMP) of the cells through either depolarizing or hyper-polarizing the cell membrane. Polarizing either restricts or facilitates release of ghrelin or other hormones through an ion channel. By artificially controlling the plasma membrane potential, hunger is regulated in direct relationship to the amount of hormone(s) restricted from or permitted to pass through the ion channel(s). In another embodiment, a pulse electric current stuns the cells of the hormone producing tissue at desired times in the metabolic cycle such as when hunger controlling substances would normally be released. After key metabolic periods have passed, hormone production is permitted to return to normal.

As seen in FIG. 2B, a second embodiment of the electrode sub-system is shown wherein a stomach 200 is provided with two sets of electrode plates 250 which are used to deliver electric current to tissue. In one example, a first plate is mounted internal to the stomach 200 or small intestine 210 and covers the area where control of the tissue is desired. A corresponding plate is mounted external to the stomach 200 or small intestine 210 sandwiching the tissue. The plates 250 may be held together magnetically, by tissue fasteners such as suture, staples, t-tags or similar or by vacuum stabilization once positioned.

As seen in FIG. 2C, a third embodiment of the electrode sub-system is illustrated wherein an array of electrode spikes 260 is pressed into the area of tissue to be controlled within the stomach 200. In one example, the array of electrode spikes 260 is arranged on a backing such that the electrodes of a particular pole are only adjacent electrodes of opposite polarity. The array of electrodes 260 is held in place with sutures, clips, staples, or other means. The electrodes may be held in place by vacuum applied between the electrode tips. The tips of the electrode spike array 260 would space the tissue off the vacuum assuring that the vacuum holes are not plugged up. In this manner, the vacuum could insure an even pressure distribution over the body of the electrodes. Furthermore, the array of electrode spikes 260 may be mounted to an implant that moves about the stomach, such as a gastric balloon or gastric coil.

It is well-known that the human body tends to fight off unwanted impurities and will reject or otherwise neutralize implant devices. Frequently, the body will encapsulate, adapt to, or otherwise diminish the usefulness of implants. Therefore a number of device and method embodiments for maintenance of the electrode systems are proposed to combat these unwanted physiological responses.

A first maintenance system includes the use of a flushing system on leads within the gastrointestinal tract (not shown). The flushing system provides a periodic flush of the functional surface of the electrode systems to prevent buildup of unwanted attenuating materials. A second maintenance system includes the use of coatings that interact with the physiological environment to change properties therein. An exemplary material for such maintenance coatings applications include hydrogels and associated polymers. A third maintenance system embeds local circuitry, such as MEMS electronics; to evaluate local tissue responses and either attenuate or amplify the signals via additional resistive, capacitive, or inductive elements. In a fourth exemplary maintenance system, redundant electrode surfaces are implanted nearby each other and a control system is used to alternately use the surfaces so as to prevent any particular area of the stomach from becoming desensitized to pulse stimulation.

In managing the aforementioned regulation systems and methods for regulation of ghrelin and/or other hormone production through electrical and/or pulse stimulation, sensing, and control systems are provided. It is desirable to have the electrode(s) activate according to natural digestive cycles in order to change the plasma membrane potential or stun metabolic hormone producing tissue. Electrodes are generally activated for a minimum amount of time to induce desired results while minimizing power consumption requirements and the body's tendency to adjust and compensate. Several embodiments are proposed herein for directing the electrodes to follow the natural digestive cycles.

In a first example of a control system, a system of electrodes that contain an array of both electrical or metabolic hormone sensors and electrical stimulator leads on the gastrointestinal system is presented that records patterns and signals of electrical or metabolic hormone activities during times of both consumption and fasting. These signal patterns are stored for reference to be played back at specific or random intervals and induce the same electrical activities or metabolic hormone release activities (through PMP regulation, reversible poration or cell stunning) during periods of consumption and fasting. This has the effect of stimulating the body into a sensation of hunger or satiety. The ability of the body to accommodate or circumvent these systems may be interrupted by programmed interval recording, re-recording signals by a physician under controlled conditions, by replaying the signals at random intervals, or other logical variations.

In an example of a method for a control system for gastrointestinal regulation, steps may include installing electrodes and other sensors for recording and playback in several locations along the gastrointestinal tract, preferably at the stomach, the duodenum, near the terminal ileum and also along several locations in between the jejunum and ileum. Recording and stimulation means for areas of the colon may be useful as well. Bi-directional communication with the electrodes is provided such that information can be transferred between the local sensors and at least one processing unit. The processing unit can be contained within one of the electrodes, in a separate implant, or external to the patient. Signals are recorded during several satisfying meals over the course of weeks wherein satisfying is defined from signatures detected in the electrical measurements or indicated by the patient and/or doctor through an interface with the processor. Recording multiple events of satisfying meals, especially over the course of therapy allows the system to have a number of signatures that can be played back to avoid the body growing accustomed to a particular pattern. Upon sensing the onset of a meal, the system can playback a set of signature signals that imitate parts or the entire set of satisfying gastric signals.

The playback can have multiple variations to prevent the body's natural accommodation. As examples and not by way of limitation, the playback may include mixed up parts of meal recordings, randomized amplitudes of signals, and various modulation envelopes. Recording times may be compressed and/or decompressed. Signals may also be sent to varying sets of electrodes. In still other examples, signals may be played backwards at varying positions throughout the gastrointestinal tract such that the signals are reversed with respect to time. Sets of signals for recording and playback can include electrical impulses delivered to portions of the gastrointestinal tract, muscular responses in the form of electrical signals and/or motion as measured by strain gages, accelerometers, etc. Playback can include the stimulus signals and mechanical stimulation via motors, piezoelectric elements, electro-active polymers, etc.

The characteristic signals may be captured as a Fourier series. A Fourier series decomposes a periodic function or periodic signal into a sum of simple oscillating functions such as mathematical sines and cosines. Once a set of Fourier series is collected, a point-by-point time interval average of the multiple series may be used to create a characteristic average series for the patient. This series or any of the original series may be played back to emulate the satiation signals for the patient. After multiple recordings, the average of the signals may be played back to the body as desired.

In a further embodiment of the control subsystem, activation of the electrodes is based on a clock internal to the system. A pre-determined activation cycle is activated based on the internal clock with cycles related to (1) estimated meal times; (2) steady time dependant rate; (3) food intake metabolic hormones, such as during sleep cycles; and (4) at random times to prevent accommodation by the body to above average levels of metabolic hormones. It is known that the body adjusts to accommodate abnormally high levels of some hormones delivered exogenously. Therefore a random schedule can be used to prevent the body from adjusting and establishing a new basis levels.

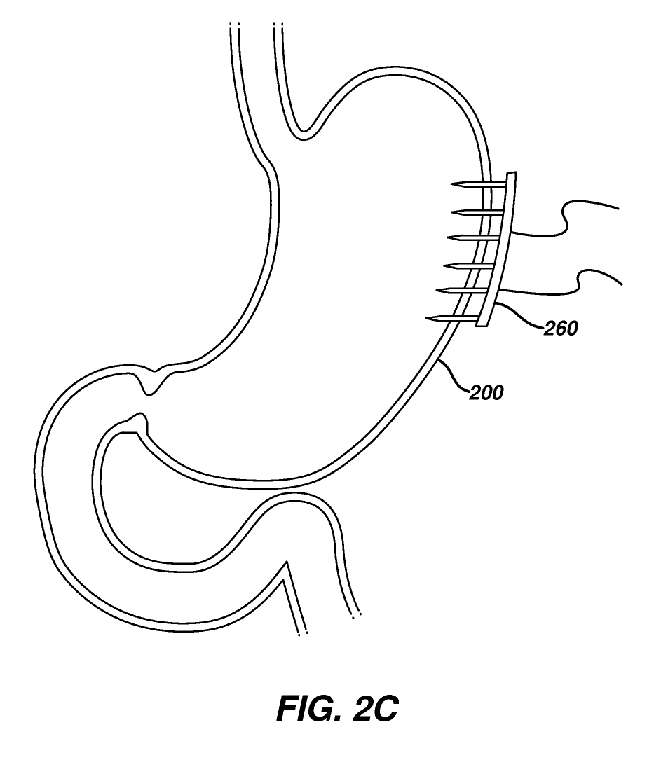

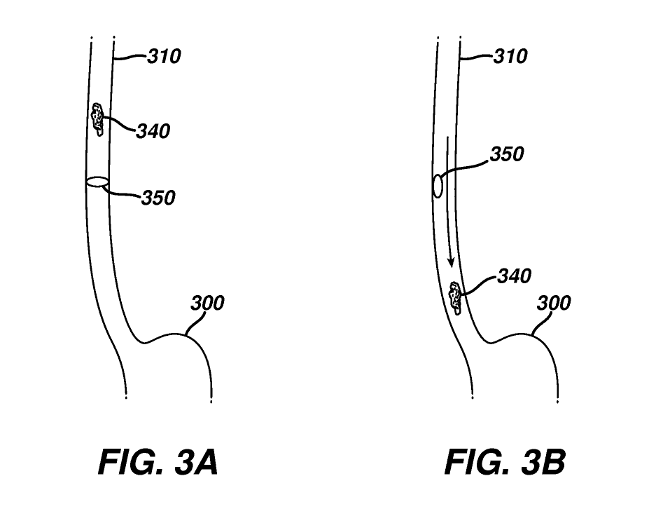

Turning now to FIGS. 3A and 3B, in a further embodiment of the control subsystem, the patient could be fitted with an implanted valve 350 that includes a sensing device. One embodiment of a sensing valve 350 is implanted in a lower portion of an esophagus 310 above a stomach 300. When food 340 is swallowed, it forces open the valve 350 in order to pass to the stomach 300. The act of forcing open the valve 350 triggers a limit switch (not shown), passing on the signal of the presence of food 340.

As an alternative, the valve may take advantage of peristaltic action of the esophagus 310 such that when a contraction or series of contractions of sufficient magnitude, frequency, or duration is detected by a strain gauge on an implanted stent (not shown) placed in the lower esophagus 310, the signal could be sent that food consumption has begun. The signal transmission device may also be advantageously placed on the stent as part of the detection system to relay this information to a receiving unit placed remotely from the stent.

Furthermore, if a patient experiences or anticipates feelings of hunger, a method of activating the electrodes is provided. As examples and not by way of limitation, such methods may include (1) a button protruding from the skin; (2) a subcutaneous button; (3) a telemetry device with remote control; (4) a change in the position of the patient such as standing, laying down, or sitting; and (5) ingesting a series of hot/cold liquids that is registered by an implanted thermocouple. Also, logic controllers can be introduced to the system to limit the frequency or intensity of patient induced activations.

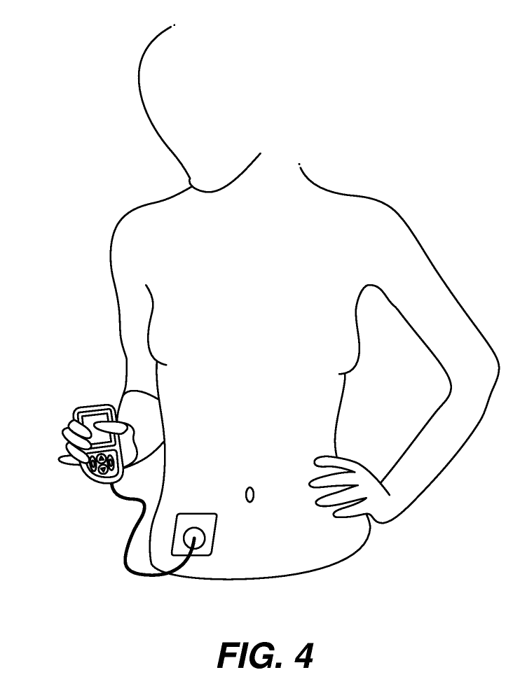

Turning now to FIG. 4, a control system including a calorie count system with a pre-programmed algorithm is shown. This system may be preferable to many obese individuals who choose to avoid invasive surgical procedures. Individuals with a metabolic syndrome typically do not recognize the relationship of caloric intake to body reaction time and indications of satiation. This is particularly true with very dense caloric foods prevalent today. Therefore there is a need for a means to enable individuals to more accurately manage their caloric intake through monitoring what they eat as through easy to use mechanisms. There is also a need for automatic closed loop appetite suppressant controls.

In a first control system, an electronic calorie counter pre-programmed with food calorie counts is programmed with new information. The device is worn by the user similar to a cell phone as seen in FIG. 4. Once a predetermined limit is reached, a vibration is provided or an alarm otherwise activated. This device quickly indicates to the individual the amount of calories consumed.

In a second exemplary system, feedback is attained using metabolic hormones. In a first example of a metabolic hormone system, feedback is achieved through measuring blood glucose dynamics for short term decline while ghrelin and leptin concentrations are measured for long term decline. These measurements are achieved via sensors placed in the stomach, intestine, and/or blood stream. In a second example of a metabolic hormone system, fat digestion, food volume/mass, and/or real time sensing of caloric intake is monitored using a sensing system. When metabolic hormone thresholds are met, a pump would inject one or more of the hormones in response to the sensed conditions.

In addition, the control system may be a weight loss tracking device to provide information about the amount of calories they have eaten in a particular meal. This system tracks caloric intake over a period of time and allows the individual to correlate this information with other meaningful data such as weight, BMI, body fat percentage, blood pressure, glucose measure, etc.

In some embodiments, the system uses a software-driven unit that interfaces with existing devices to collect and store data. The user inputs initial values for weight, height, etc. Before each meal, the person places their food on a scale that measures its weight. The user then locates the food in a database, so that the relative proportions of fat, protein, carbohydrates, and sugars may be computed and an accurate estimate of the caloric value of the meal may be calculated. The Newline Digital nutrition scale model SAD4181-SL available from Mii Wintime International, Inc. of Hicksville, N.Y. is one example of a basis for measuring caloric intake.

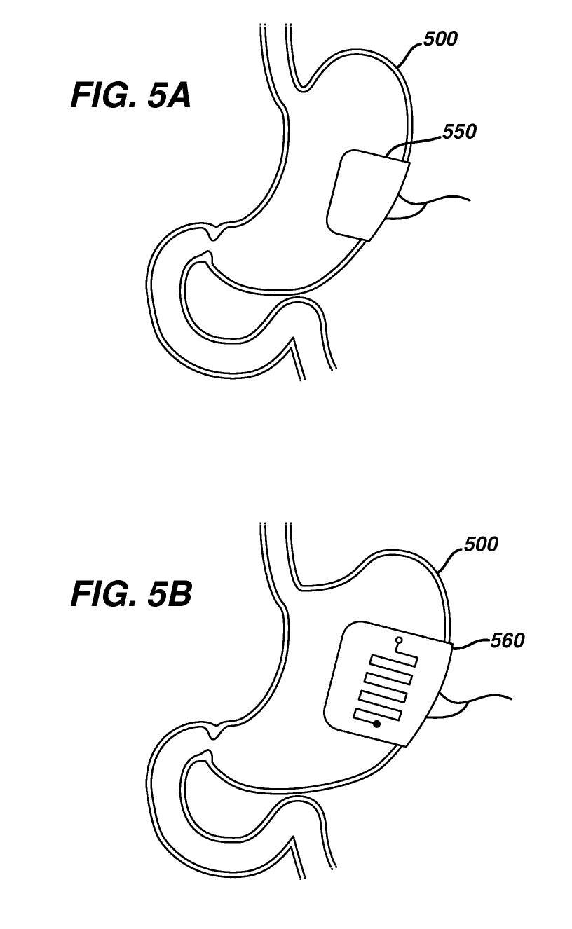

Turning now to FIGS. 5A and 5B, in other control methods, systems are used to detect stretching of a stomach 500 due to the presence of food. Measurement sensors may be strain gauges 550, 560 mounted on a coil which detect movement of the coil as a threshold strain is reached. Alternatively, a Hall Effect sensor or proximity sensor is used to detect motion of substantial magnitude to indicate that a significant event has occurred. In other sensor examples, pressure sensor detectors are mounted in proximity to the stomach or ring/band sensors are mounted in proximity to the gastrointestinal tract. In yet another example, a gastric band is fitted with a strain gauge such that when food is in close proximity to the band, the band stretches and provides a sensory trigger to the strain gauge. A strain gauge could be attached to the stomach wall. The strain gauge may be sutured to the external wall of the stomach using a laparoscopic or endoscopic procedure. In another embodiment, as food contacts tissue in close proximity to a gastric band, forces are transmitted to the band, which increases the pressure of the fluid in the band. A pressure sensor inside the band senses the change in pressure and sends a signal to the control system.

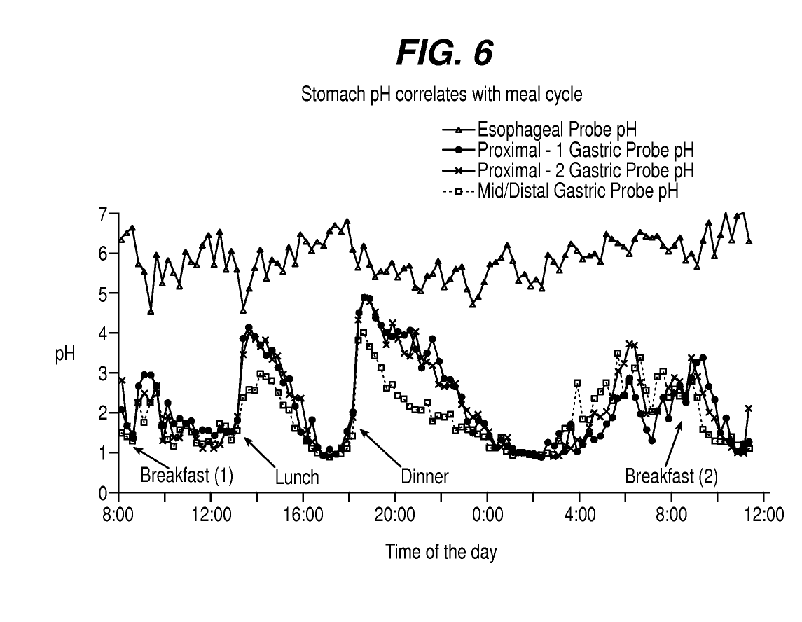

In still another embodiment, a pH sensor placed inside the stomach is used to indicate meals cycles. As seen in FIG. 6, stomach pH levels strongly correlate with meal cycles received by a patient during the day. The pH sensor is set with threshold levels which cause activation of a weight loss system through electrode systems described herein. Ingested food is held in the stomach and treated with bile and acids. A pH microsensor in the stomach identifies when the pH is increasing and sends a reversible electroporation signal to the pylorus that provides some relaxation of sphincter muscles. Relaxation of the sphincter muscles allows some content release. Other signals stimulate peristalsis. Hyperperistalsis activity moves the bolus through the digestive system more quickly to produce GLP and satiation effects sooner.

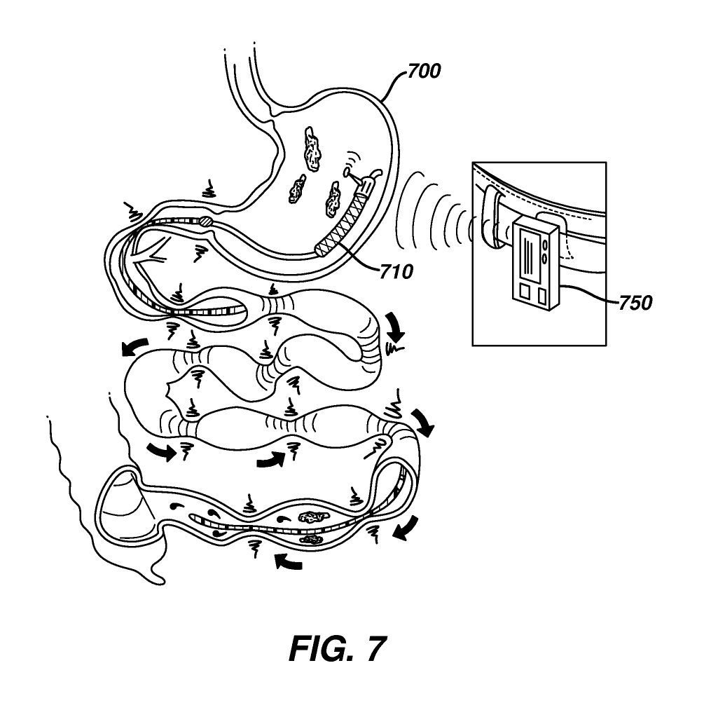

Turning now to FIG. 7, several means are presented for recharging the system presented as examples and not by way of limitation. The system includes a pH sensor 710 in a stomach 700 and may be powered by a rechargeable transceiver 750 worn during waking hours in some embodiments. Alternatively, the transceiver 750 may be worn during sleeping hours. Additionally, the peristaltic activity of the stomach may be used to activate an energy harvesting device which recharges the batteries.

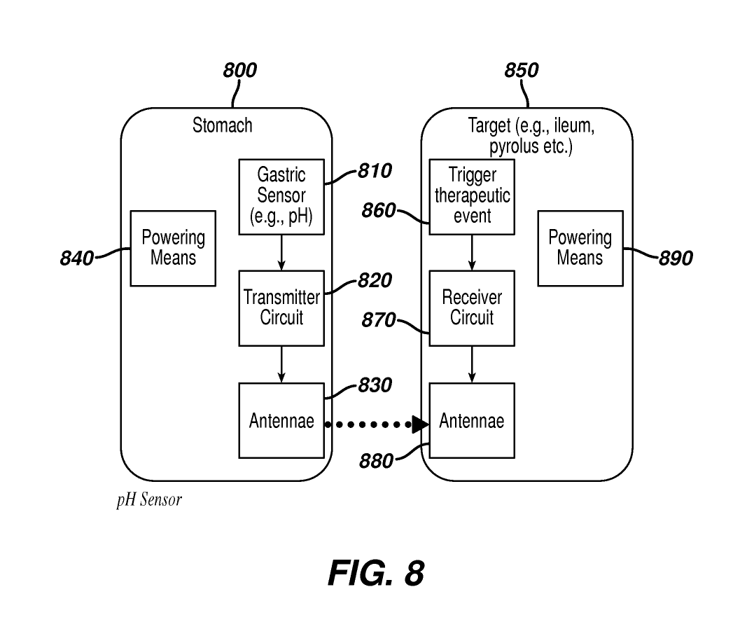

Turning now to FIG. 8, a schematic of a subsystem is shown for triggering a reversible electroporation signal to a targeted area. As shown schematically implanted into a stomach 800, the system includes a gastric sensor 810 such as a pH sensor having transmitter circuits 820 operatively connected to antennae 830. The system may also include powering means 840. When pH thresholds are achieved, a therapeutic event 860 is triggered at a targeted area (e.g. ileum, pyrolus 850) that is transmitted to receiver circuitry 870 to initiate a response by means of an antenna 880. Again, the system may optionally include powering means 890.

Weight loss is managed by initiating treatment at the onset of a meal to create a feeling of satiation faster. The treatment is not constant, but is preferably initiated by signals from the gastric cavity indicating a meal has started. A simple trigger can be a change in intragastric pH. The use of triggered reversible electroporation signals makes the body less likely to adapt to variable and unpredictable stimuli.

In an alternative embodiment, a marker-based activation may be used. For example, it is known that ghrelin cycles anticipate meals as well. A sensor capable of detecting ghrelin or other markers which are tied to the prandial period may be used as a sensing system. In one embodiment, a system such as is described below would suffice to detect ghrelin, which is released into the blood system. The system may be placed in fluid communication with the blood stream to determine ghrelin or other peptide/protein levels.

Another embodiment for control mechanisms includes a triggering pill swallowed with meals or at times of increased hunger. The pill includes mechanisms used to activate the electrodes. In an example of a triggering pill, the pill is a coated iron pill and includes a magnetic sensor. The coating may comprise a biocompatible substance. When the patient eats a meal, the pill is swallowed. When the pill enters the stomach, sensors within the implanted device detect its presence using magnets, Hall effect sensors, or similar mechanisms. When the pill is sensed, a predetermined activation of the electrodes is initiated.

In another example of the triggering pill, the pill emits an intermittent sonic pulse or electrical pulse that is detected by the electrode setup. The sonic emission may be at a frequency that advantageously penetrates air, ranging from 20 Hz to 40 kHz. Additionally, the pill may use visible light range, ultraviolet, near infrared, or far infrared pulses that are detected by the electrodes as an activation signal. As an alternative, the pill may emit microwave or any other electromagnetic pulses that are detected by the electrode.

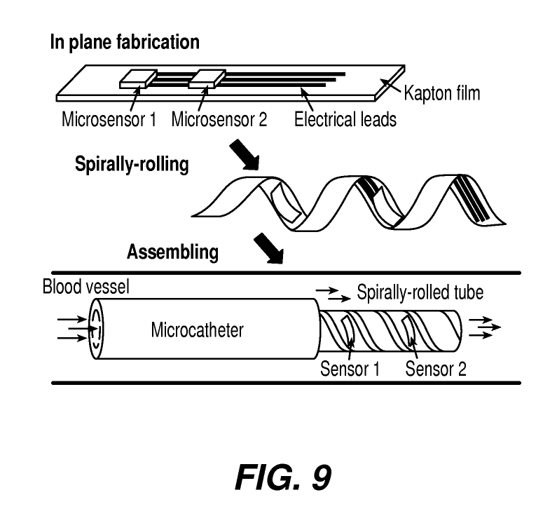

In another embodiment for using a sensing device to trigger an actuator as seen in FIG. 9, flexible biosensors are deployed internal or external to a body lumen along the gastrointestinal track such as at the ileum. The sensor detects glucose or fat which are used as a trigger based on predetermined threshold levels. The increase or decrease in nutrients can be used as a trigger point for a pump, stimulation devices, and the like. As an example, the sensor may be wrapped around the internal upper portion of the stomach to detect food intake into the body.

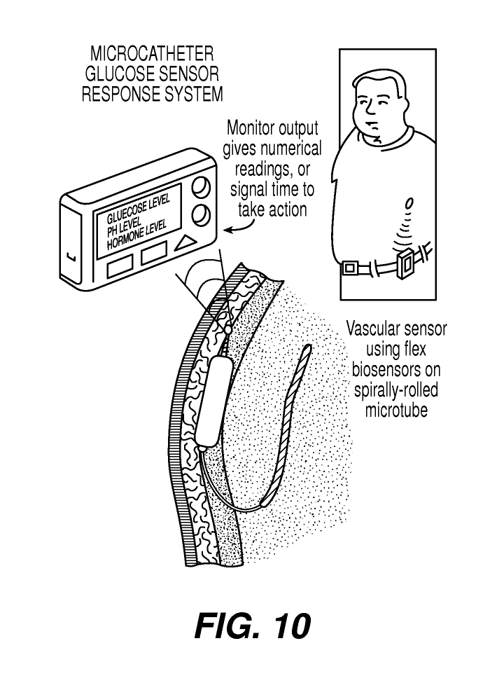

Implantation of a vascular sensor can be used to monitor glucose levels of any detectable hormone, chemical, physiological indicator (e.g. pH) with a receiving system of a personal type such as surgically implanted components or worn components (belts, pendants, watches, etc). Transceivers allow continuous monitoring of subject measurement and these transceivers may be wireless, rechargeable, passive, or active. Monitored output may give numerical readings or signal time to take an action. Such actions may relate to food, pills, insulin, and other appetite influencing mechanisms and may include sounding an alarm. Such responses may be to any combination of sensed or detected activity. The sensor could also be used to automatically trigger a response and take the appropriate action. Sensors may be used in combination with submucosal implants as illustrated with respect to FIG. 10. Such sensor and implant systems offer noteworthy benefits in that they can trigger a satiation event earlier than the body would normally initiate, maintain constant satiation levels, and maintain constant feedback loops for controlled actuation of regulating devices.

In another embodiment of a control system, a user interface is employed. The interface includes equipment that permits the patient to visualize sensor data such as from blood sugar levels, hormone levels, stomach motility, gastrointestinal track speed, other stimulator feedback such as stimulation levels, functional level, time until next service needed, and the like. The control system may include a feedback device that accesses data from the sensed stimuli and/or displays real-time data retrieved and acted upon by the sensor systems. The user interface works in conjunction with the control system to deliver better information to the control system. As an example, the patient may enter the time a meal starts and what is eaten. The control system then compensates for the meal and delivers the appropriate amount of treatment to offset the meal. The control system may also contain a processor and memory to serve as a record-keeping device to assist with compliance.

In another embodiment of the sensing system, the system senses the amount of metabolic hormone (e.g. GLP-1) in the body and determines the level of activation for the electrodes in direct relation to the amount of metabolic hormone present. For example, a MEMs sensor may be used including an array of cantilever beams with DPP4 or a surrogate binding peptide as a surface coating embedded within a chamber with a nanomembrane cover in which interstitial fluid passes through. The beams are tuned to resonate at a certain frequency and would be electrically driven. The presence of GLP-1 will bind to the DPP4 and change the mechanical properties of the beam thus changing the resonant frequency. The degree of change would be an indicator of how much GLP-1 was present. The beams could also be pulsed to raise the temperature at periodic intervals to denature the GLP-1 bond and reset the sensor. The nanomembrane diffusion barrier is designed to minimize the noise in the interstitial fluid containing a number of different hormones by only allowing the GLP-1 hormone to pass through. Any resonant structure appropriately sized to be sensitive to small changes in mass will suffice. Examples of such structure includes PZT disks vibrating in their thickness mode, PVDF with a curve, unimorphs, biomorphs, and magnetostrictive structures and films as examples. Pairs of cantilevers where one is coated with specific vectors to monitor particular substances are also useable.

These types of sensing and control systems offer several benefits in practice to those of ordinary skill in the art. A first benefit is the ability to measure hormones such as GLP-1 in vivo with a high degree of accuracy and sensitivity in real-time. An additional benefit is the ability to readily determine the effectiveness of therapy and treatment. Another benefit is the ability to interactively communicate status enabling a manual or automatic response.

In still other embodiments, several sensors together could be used to trigger the control system and activate the system. Alternatively, an external device on the outside of the body is used to trigger the start of a meal. The device sends a signal to the sensor control system. The control system then activates a predetermined response that interacts with other control systems to give benefit to the patient.

Power input and storage systems are also an integral part of using electrodes to induce plasma membrane potential effects in metabolic hormone producing tissue. Several embodiments for power systems are described herein.

In a first power system embodiment, power is stored using a battery. The battery is positioned inside the stomach, inside the body cavity, or outside the body. Intragastric placement of the battery may use endoscopic or laparoscopic surgical procedures. Alternatively, a replacement battery may be swallowed as a pill. The casing of the battery may be selectively magnetic as in the mounting base for a dial indicator.

In an example of a battery power supply system, a base is made from two blocks of iron, with a round cavity bored through the center. The halves are joined together with a non-ferrous material such as brass or aluminium. A round permanent magnet is inserted into the bored hole and a handle is attached to allow easy rotation of the magnet. Rotation changes the direction of the magnetic field so that it is either directed into the two halves, where the iron blocks act as keepers (“off” position), or directed so that the field traverses the non-ferrous material between the two halves (“on” position). In the “on” position, the field is effectively passing across an air gap where it is made to do work, if this gap is bridged with another piece of iron, it becomes part of the magnetic field's circuit and is attracted with the full strength of the magnet to provide a clamping effect.

A passive, magnetic base can therefore be attached in a variety of positions to any attractive surface, allowing the base to be positioned in an optimal orientation for the part to be tested. Combined with the flexibility of movement allowed by the arms gives the operator a large range of options in positioning the dial indicator. A battery constructed on these principles can dock to a magnetic base in the gastrointestinal tract in the desired orientation. Contacts on the battery casing contact the appropriate contacts on the implanted base unit and electrical connectivity is established. As the battery approaches low charge, a signal is sent to an outside receiver of a release of the battery unit for passage through the gastrointestinal tract. The battery is released as the core rotates 90 degrees and the battery becomes non-magnetic relative to the passive base, allowing the battery to move away from the base.

In a second embodiment of a power system, electromagnetic induction is used to pass electrical energy through the tissue without disturbing the patient. In a first example, the implanted device includes a conducting coil. A pack may also be worn by the patient including the conducting coil driven by batteries. The magnetic field created by the coil in the pack would cause a current in the implanted coil, which would drive the implanted device. In another example, the implanted device includes a coil and battery. The patient periodically places an external coil against the skin and the external coil is powered by an external power source. In a third example, a coil triad comprised of three coils disposed at 120 degrees relative to each other such that one coil or a vector combination of two coils are positioned relative to the external coil allowing power to be inductively transferred by transcutaneous energey transfer (TET). An exemplary embodiment of such a device is shown in “Means for Determining the Position of the Sensor” U.S. patent application Ser. No. 12/043,230 filed Mar. 6, 2008, which is incorporated by reference in its entirety.

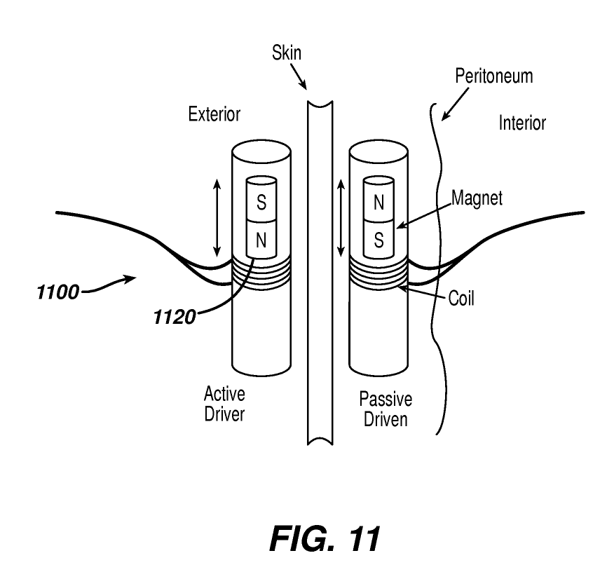

Turning now to FIG. 11, in a third power embodiment, a coil or magnet assembly 1100 is implanted within the subdermal layer of the skin. An external powered oscillating magnet is placed in close proximity to the patient's skin. A driver magnet 1120 oscillates the driven magnet such that the changing magnetic field produces electricity.

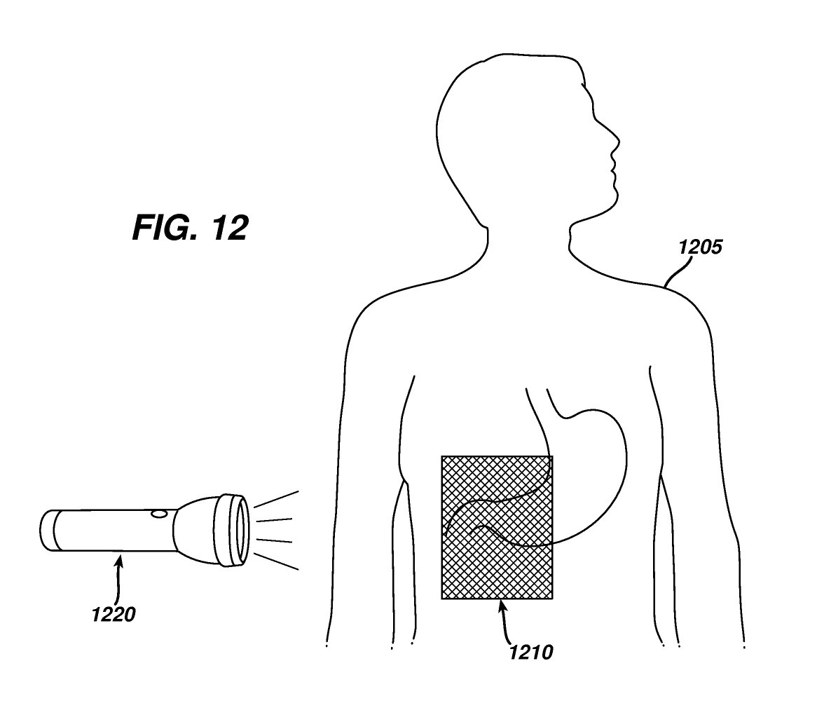

In a fourth powering embodiment seen in FIG. 12, infrared light is used as a power source. A subcutaneous photovoltaic 1210 is first implanted below the surface of the skin at a desired location within the body of a patient 1205. An infrared light source 1220 is then used to penetrate several layers of tissue to provide a recharging source of power to the cell 1210.

In a fifth set of power subsystem embodiments, devices are disclosed that recharge using the body's own energy. Such recharge capability has the potential to prevent the need for expensive, time consuming and invasive power replacement needs. Two non-limiting exemplary embodiments for devices that harness the energy of the body to produce electrical energy are proposed. In a first example, subtle movements by nanowires are used to generate electricity using the piezoelectric effect. The mechanical stress produced by bending a zinc oxide nanowire creates an electrical potential across the wire. This potential drives current through a circuit. The conversion of mechanical energy to electrical energy is the piezoelectric effect and may be created by natural motions within the body. In an illustrative example, these wires are bundled and attached to the patients bone at a joint.

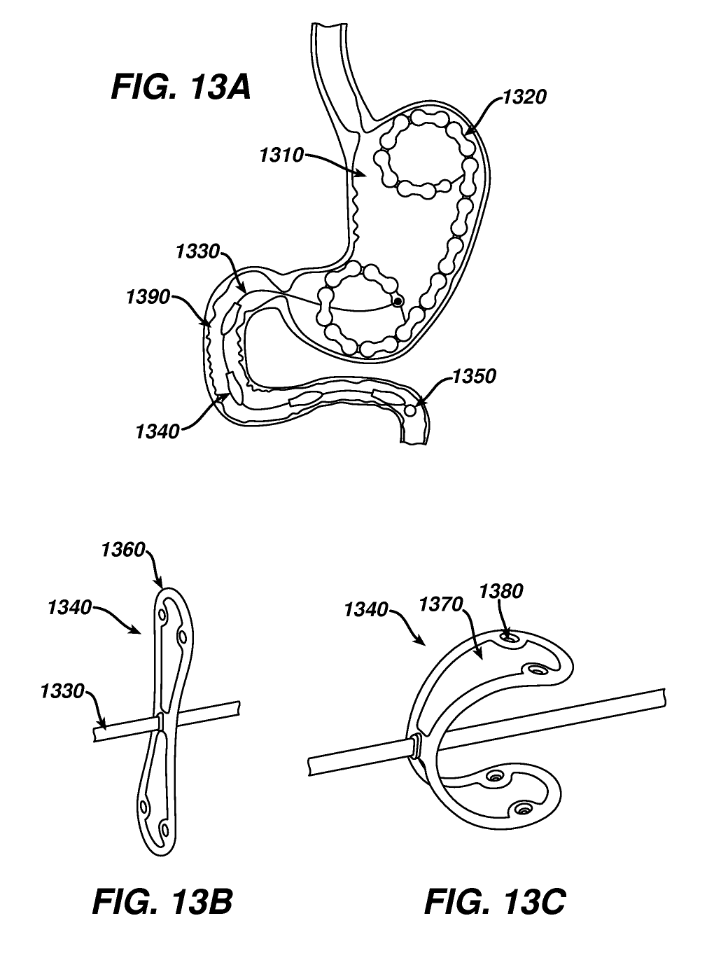

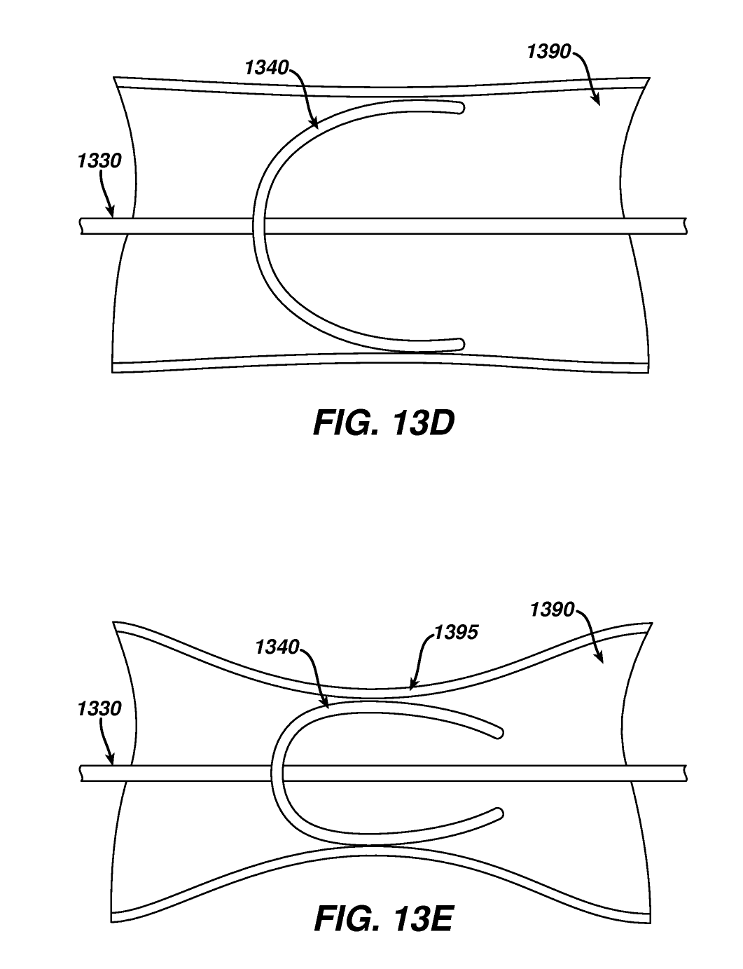

Turning now to FIG. 13A, in a second example of using the body's internal energy as a powering system. A gastric coil 1320 is endoscopically inserted into a gastric cavity 1310 having a mucosal lining of the duodenal lumen 1390. A rechargeable battery or series of batteries are located on or in the gastric coil. A wire lead 1330 is run in series with the batteries. The wire lead is propelled into the duodenal lumen with peristaltic motion working on leading sphere 1350. Energy generating petals 1340 are placed along the wire and are run in series electrically. The petals 1340 are made of a material that generates electricity when flexed such as with a piezoelectric fluoropolymer film, such as DT1 and SDT1 manufactured by Measurement Specialties of Hampton, Va. An electric charge is delivered to the batteries when peristaltic activity bends the individual petals 1340.

FIGS. 13B and 13C illustrate energy generating petals 1340 along the wire lead 1330. A piezoelectric fluoropolymer film 1370 is encased in a soft elastomeric material 1360 that allows for flexing yet withstands the gastrointestinal environment. An example of a suitable material is santoprene. The elastomeric material 1360 protects the mucosal lining of the duodenal lumen 1390. The holes 1380 may be used to secure the petals 1340 into a compressed position during insertion by placing an absorbable rivet between the holes 1380 on opposite ends of the petal 1340. The rivet could be made of any absorbable material known in the art such as Vicryl or PDS.

FIG. 13D shows the energy generating petal 1340 inside the duodenal lumen 1390 in a moderately flexed position. FIG. 13E shows the energy generating petal 1340 in a more flexed configuration as it is being acted upon by a peristaltic wave 1395 within the duodenal lumen 1390.

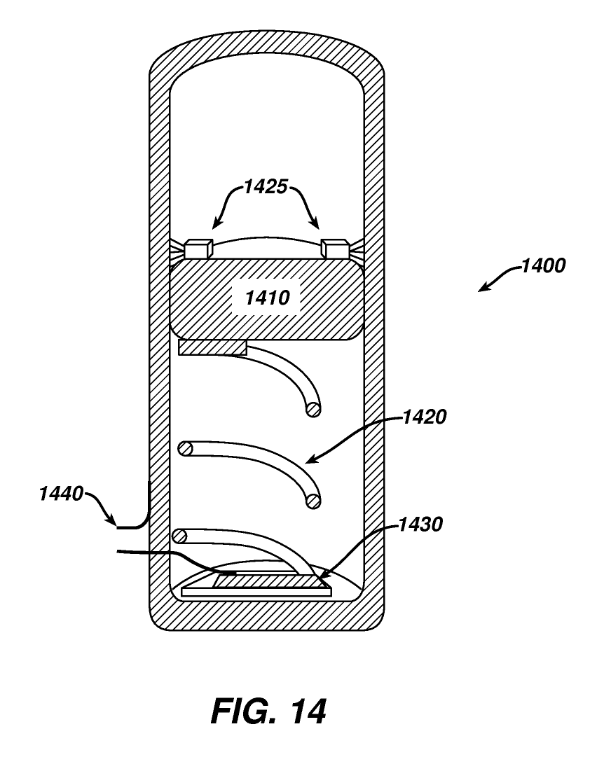

In another power system embodiment shown in FIG. 14, a mass/spring/damper system is used to generate energy from the natural motion of the body. The resonant frequency of the system is designed to match the natural walking gate of a patient. As the patient walks, the system is driven to oscillate, and energy is harvested from the oscillating system. One embodiment of the system generates electrical power, and is shown in the figure below. The invention described below uses a mass/spring/damper system to generate energy from the natural motion of the body. The resonant frequency of the system is designed to match the natural walking gate of a patient. As the system is driven to oscillate, energy is harvested from the oscillating system. In some embodiments, the system generates electrical power.

In the FIG. 14, a cylinder 1400 is implanted in the patient such that its orientation aligns with the natural walking oscillation of the body. Within the cylinder 1400, a mass 1410 is connected to a piezoelectric spring 1420. The spring 1420 is held in place by an insulating material 1430. The spring 1420 is in electrical communication with the mass 1410, which is in electrical communication with the inside of the cylinder 1400 due to sliding brushes 1425. When the patient walks, the system oscillates. The oscillation deflects the spring 1420 that causes an electric potential between wires 1440 of the system. The electric potential is used to power implanted devices.

Another embodiment similar to that shown in the FIG. 14 uses a magnetic mass and coil to generate electric potential. The coil is wound around the axis of the cylinder. When the mass oscillates due to movement of the patient, current is generated within the coil. The current is used to power implanted devices.

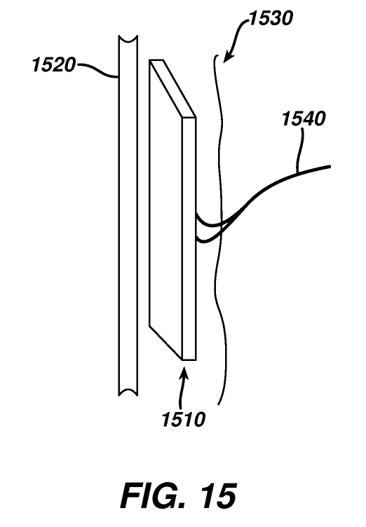

Turning now to FIG. 15, an alternative embodiment is shown having a “Peltier” or thermal electric device 1510 placed just below the patient's skin 1520 above the peritoneum 1530 and attached electrically by wires 1540 to the port. The device is charged by placing a cool object on the surface of the skin 1520 just above the device 1510. Electricity to charge the port is generated from the thermal differences between the body and the cool substance.

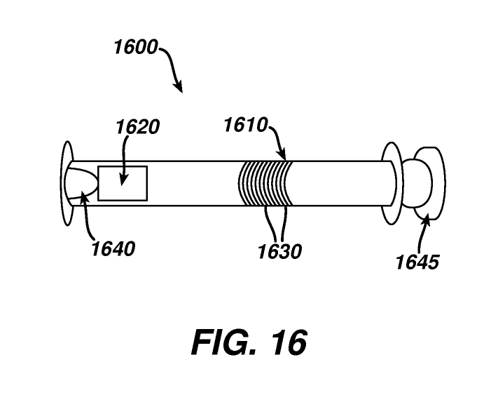

Turning now to FIG. 16, a mechanism 1600 that converts mechanical energy into electrical energy using a coil 1610 and sliding magnet 1620 is placed within the body in order to power the system, or charge the battery. The mechanism produces power from simple patient motions like walking via wires 1630 and may optionally include front and rear bumpers 1640, 1645 to restrict motion of the magnet 1620. The mechanism is generally oriented to derive maximum translation from walking, driving, or other bodily acceleration. Energy generated by simple mechanical motions could be captured for later use by storage means such as by internal or external batteries.

Further embodiments of the power subsystems may be a smart coil powering galvanic powering means deriving power from the gastric environment, from internal or external kinetic energy generation or from thermal energy sources. These are described in “POWERING IMPLANTABLE DISTENSION SYSTEMS USING INTERNAL ENERGY HARVESTING MEANS” U.S. patent application Ser. No. 12/261,089 filed Oct. 30, 2008 [END6518] which is incorporated by reference in its entirety. Additionally, power generated by internal or external motion of the body may be used to. One example is use of compressions of a gastric coil to charge a ratcheted torsion spring to continue winding of the spring until the spring reaches its mechanical limit. The ratchet pawl may be released on demand to cause unwinding and thereby release the mechanical energy stored within the spring. This may be converted to electrical energy by coupling the spring with a coil and magnet assembly. The energy may be stored in a battery or used on demand.

Mounting and fixture systems are also employed to prevent the electrodes, control systems, and power storage systems from becoming dislodged due to paristolsis or other forces caused by the body. Several embodiments of mounting and fixture devices and methods are disclosed herein.

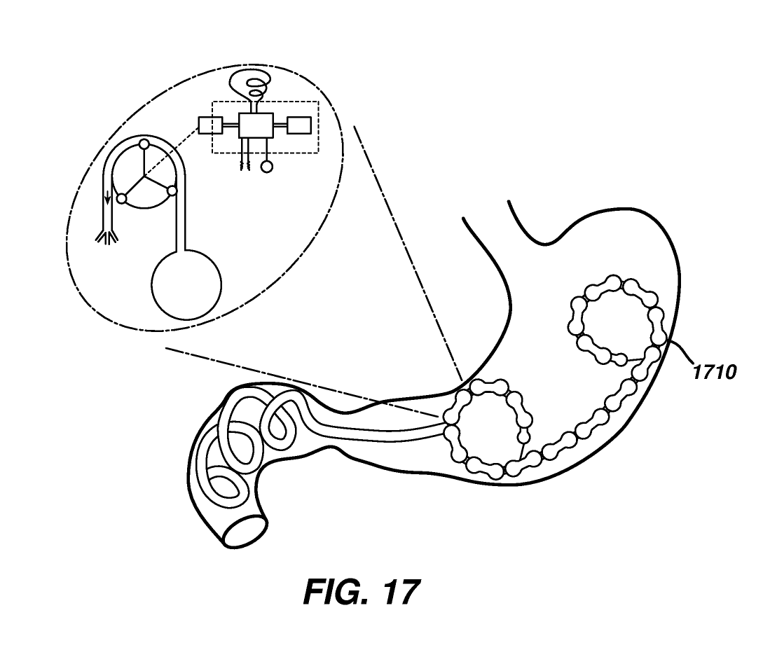

In a first mounting embodiment of FIG. 17, a gastric coil 1710, as described in U.S. Patent Application WO 2008/028108 A2, is endoscopically inserted into a gastric cavity as described in the above mentioned references. The device senses a physiological change associated with food ingestion or hunger and provides a mechanism adapted for direct stimulation of a region responsive to gastrointestinal satiety agents. In addition to releasing substances capable of stimulating a gastrointestinal satiety trigger, the device itself may expand to trigger stretch receptors of the stomach. Use of gastric coils are relatively easy to deploy and remove and are generally well-tolerated by the body during the time of implantation.

In a second mounting embodiment, components of the system external to the stomach are mounted on a gastric band. Since most gastric bands are implanted laparoscopically, the mounting of these components can be similarly performed in a minimally invasive procedure. Alternatively, portions of the system may be mounted on a gastric band catheter or on a refill port placed subcutaneously on a fascia layer.

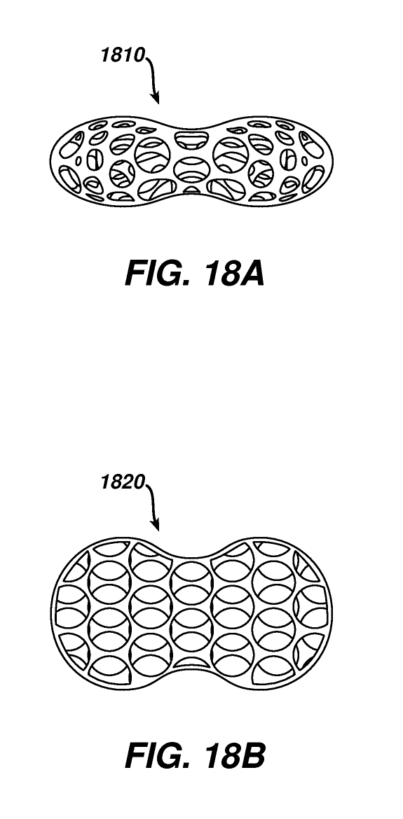

Turning now to FIGS. 18A and 18B and in a third mounting embodiment, a belly ball 1810, 1820 is presented. The belly ball 1810, 1820 is a collapsible spherical, oblong, or football-shaped cage that is inserted into the stomach endoscopically or laparoscopically through the esophagus in a collapsed form 1810, and then expanded to an expanded form 1820 once inside the stomach. The ball 1810, 1820 is large enough to prevent passage through a pyloric sphincter and provides a platform or anchor for devices attached to it that are intended to remain in the stomach or at some fixed location beyond the stomach (such as via a tether). The ball 1810, 1820 may also provide an anchor for a second, smaller ball via a tether.

The purpose of the devices attached to the belly ball may be to measure or monitor conditions or substances within the digestive tract, or to provide therapeutic effects (e.g., delivery of drugs, hormones, or electrical stimulus, altering of pH, manipulation or in-vivo manufacture of hormones or other substances, etc.). They may incorporate diagnostic or computing capabilities (e.g., lab-on-a-chip), and provisions for communicating with devices or instruments outside the body (by wireless or other means). They may be powered by various means, including direct or wireless transmission, magnetically coupled resonance, stored energy, ambient energy harvesting (mechanical, electrical, chemical, acoustical, etc.), biologically-generated energy, etc.

In a more rigid, robust form, the ball itself may also have a direct mechanical and therapeutic effect on the digestive system by affecting the mechanisms of satiation and/or satiety, or by altering the normal digestive processes that occur in the stomach (e.g., by altering the normal secretion of gastric juices, lessening or disrupting the mechanical mixing of these juices with the food, delaying the release of chyme into the duodenum, etc.). The ball may also be designed such that its size and/or shape is adjustable in response to inputs by any of a number of influences. As an alternative to or in conjunction with a cage-type construction, the framework of the ball may incorporate tensegrity structures.

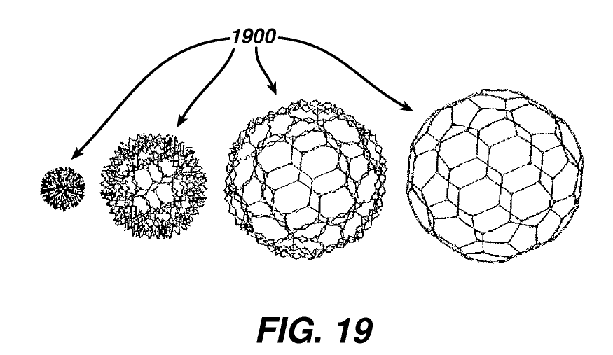

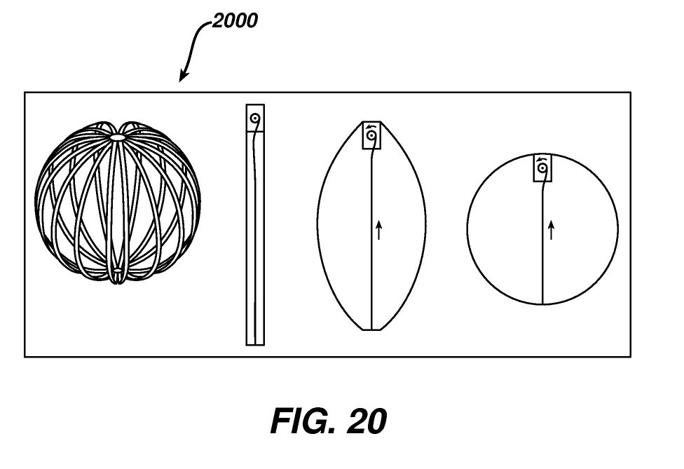

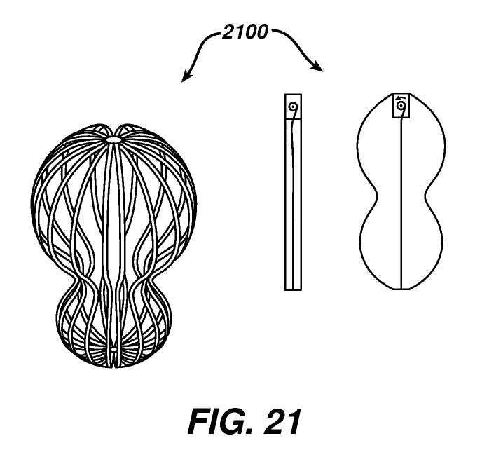

Expansion of the ball in the stomach is accomplished passively (spring into shape), or actively via self-contained or externally-applied mechanisms. Mechanically, the framework for expansion and contraction of the ball could employ various flexible, elastic, folding, bending, rotating, twisting, sliding, or pivoting elements such as with a Hoberman sphere 1900 shown in FIG. 19. Other configuration examples are shown as the spherical flex-ribbed structure 2000 of FIG. 20 and in the asymmetric oblong flex-ribbed structure 2100 of FIG. 21. For removal, the ball may either be designed to be re-collapsed and removed through the esophagus, or could be designed to re-collapse or dissolve after a given time and exit the body via the small and large intestines. The collapsible ball concept may also be used for measurement or therapeutic purposes in other cavities within the body.

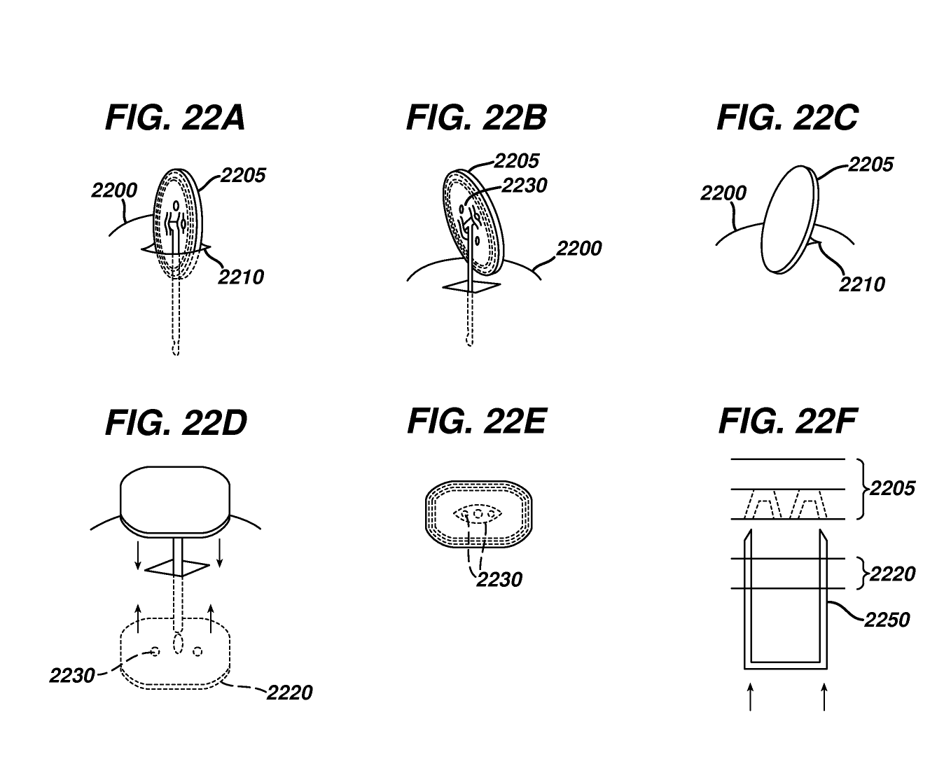

Turning to FIGS. 22A-F, in a fourth mounting embodiment, an anchor point is inserted into a stomach 2200 through the esophagus and attached to the stomach wall. One means of attaching the anchor point to the stomach wall involves the use of a stapler anvil 2205 that is inserted through an incision 2210 in the stomach wall. After stapling, the anvil 2205 remains a part of the anchor point as seen in FIG. 22C. This anvil 2205 has a pivoting, hinged stem, which either through its cross-sectional shape (e.g., oval) and/or by one or more protrusions on its sides, is used to orient the anvil 2205 rotationally from inside the stomach 2200 once the anvil 2205 is deployed. A backer plate 2220 having through holes 2230 through which the staples pass is then aligned to the anvil 2205 via spring-loaded alignment pins (not shown) in the stapler that pass through the backer plate 2220, through the incision, and into blind holes in the anvil. The anvil 2205 and backer plate 2220 are drawn together by the stapler as seen in FIG. 22D. The stapler is then actuated forming the staple legs 2250 into the anvil 2205, and securing the stomach wall between the anvil 2205 and the backer plate 2220. If desired, multiple frame/anchor pairs could be employed.

In other embodiments, the internal frame is eliminated, and two or more anchor points are attached within the stomach wall, then either fastened together inside the stomach to mechanically constrict it, or mutually drawn together and fastened to flatten it. In still other alternative embodiments, the hinged rigid stem is replaced by a flexible member, such as a cable or chain.

In still other embodiments of fixing and mounting means, a needle pierces the stomach wall and a grasper is introduced through a trocar to attach the stem to the abdominal wall. Further, a T-Tag could be used to place an anchor into the stomach wall. First a suction cup is used to pull tissue into the cup with vacuum then the T-Tag is pierced into the tissue in the center of the cup area and T-Tag deployed. This method protects the tissue outside the stomach from blind needle sticks. In another example, the attachment means is a spiral fastener which corkscrews into the stomach wall for secure fastening. The tip of the spiral fastener may have a retrograde barb to prevent the spiral from being easily released from the wall of the stomach. Devices may be mounted on stents and placed anywhere in the gastro-intestinal tract. Also, components of the system external to the stomach could be held in place using sutures. Laparoscopic suturing methods permit minimally invasive implantation procedures.

One skilled in the art will appreciate additional features and advantages of the invention based on the above-described embodiments. Accordingly, the invention is not to be limited by what has been particularly shown and described, except as indicated by the appended claims. All publications and references cited are expressly incorporated herein by reference in their entirety.

I/we claim:

1. A method for regulating production of a hormone, the method comprising:
a. placing at least one electrode in a gastrointestinal tract of a patient;
b. recording an electrical signal during a preselected event produced by the gastrointestinal tract;
c. storing said electrical signal; and
d. playing back said electrical signal by activating said electrode during the absence of said preselected event.

2. The method of claim 1 wherein said step of recording an electrical signal during a preselected event produced by the gastrointestinal tract comprises activating a recording capability of said electrode.

3. The method of claim 1 further comprising implanting an electrical signal recording device.

4. The method of claim 1 wherein said at least one electrode is placed at a location selected from a stomach, a small intestine, a duodenum, a terminal ilum, between a jejunum and an ileum, a colon, or a combination thereof.

5. The method according to claim 1 wherein said preselected event is selected from a point in time of a metabolic cycle of said patient, activation of a digestive cycle in said patient, a fasting state, a consumption state, and combinations thereof.

6. The method of claim 1 wherein said playing back step is based on a predetermined activation cycle activated based on an internal clock having a cycle selected from estimated meal times, a steady time dependant rate, food intake metabolic hormones, such as during sleep cycles, random times, or a combination thereof.

7. The method of claim 1 wherein said step of playing back said electrical signal modifies a plasma membrane potential of a cell, wherein said electrical signal creates a pore in said cell sufficient to allow passage of said hormone through said pore.

8. The method of claim 1 wherein said step of playing back said electrical signal results in the formation of an electrical field, wherein an electric potential is limited to the range of about 0.0 to about 1.0 volts.

9. The method of claim 1 wherein multiple sites are used to administer multiple magnitudes of electric potential.

10. The method of claim 1 comprising the step of providing a sensing device, wherein said sensing device is capable of detecting feedback selected from a metabolic signal such as glucose, fat, ghrelin, leptin, and nutrients, stretching of a stomach due to presence of food, motion, pressure, contact of food with a preselected location, pH, presence of a triggering pill swallowed with meals or at times of increased hunger, and combinations thereof.

11. The method of claim 1 wherein the act of activating said electrode performed by said patient using a mechanism selected from a button protruding from said patient, a telemetry device with remote control, a change in a position of said patient, ingestion of hot and/or cold liquids capable of being registered by an implanted thermocouple by said patient, logic controllers, a calorie count system, and combinations thereof.

12. The method of claim 1 wherein said method provides satiety to said patient.

13. The method of claim 1 wherein said at least one electrode is selected from a two electrode spike, two sets of electrode plates, an array of electrode spikes, and combinations thereof.

14. The method of claim 1 further comprising the step of providing at least one of a flushing system on leads within the gastrointestinal tract; coatings that interact with the physiological environment to change properties therein; embedding of local circuitry; alternating electrode surfaces; or combinations thereof.

15. The method of claim 1 wherein said hormone is selected from ghrelin, gastrin, somatostatin, secretin, cholecystokinin, a CCK analog, a CCK receptor agonist, incretins, GLP-1, GIP, DDP-4, ghrelin, ghrelin antagonist, leptin, neuropeptide Y, peptide YY, a PYY analog, GLP-1, a GLP-1 analog, oxyntomodulin, cortisol, deoxycorticosterone, flurohydrocortisone, beclomethasone, betamethasone, cortisone, dexamethasone, fluocinolone, fluocinonide, fluocortolone, fluorometholone, fluprednisolone, flurandrenolide, halcinonide, hydrocortisone, medrysone, methylprednisolone, paramethasone, prednisolone, prednisone, triamcinolone, danazole, fluoxymesterone, mesterolone, dihydrotestosterone methyltestosterone, testosterone, dehydroepiandrosetone, dehydroepiandrostendione, calusterone, nandrolone, dromostanolone, oxandrolone, ethylestrenol, oxymetholone, methandriol, stanozolol methandrostenolone, testolactone, cyproterone acetate, diethylstilbestrol, estradiol, estriol, ethinylestradiol, mestranol, quinestrol chlorotrianisene, clomiphene, ethamoxytriphetol, nafoxidine, tamoxifen, allylestrenol, desogestrel, dimethisterone, dydrogesterone, and combinations thereof.

16. A method for regulating hormone production, the method comprising:
a. placing at least one electrode in a gastrointestinal tract of a patient;
b. recording a first electrical signal during a preselected event produced by the gastrointestinal tract;
c. storing said first electrical signal and creating a second electrical signal by modifying said first electrical signal; and
d. playing said second electrical signal by activating said electrode during the absence of said preselected event.

17. The method of claim 16 wherein said step of modifying said first electrical signal comprises reversing the time order of said first electrical signal.

18. The method of claim 16 wherein said step of modifying said first electrical signal comprises amplifying said first electrical signal.

19. The method of claim 16 wherein said step of modifying said first electrical signal comprises time scaling said first electrical signal.

20. A method for regulating hormone production, the method comprising:
a. placing at least one electrode in a gastrointestinal tract of a patient;
b. providing at least one sensor;
c. recording an electrical signal during a preselected event produced by the gastrointestinal tract;
d. storing said electrical signal; and
e. playing back said electrical signal by activating said electrode in response to a signal received from said sensor.
